(19) United States
(12) Patent Application Publication
(10) Pub. No.: US 2012/0184590 A1
(43) Pub. Date: Jul. 19, 2012

(54) FORMULATIONS OF INDOLE-3-CARBINOL DERIVED ANTITUMOR AGENTS WITH INCREASED ORAL BIOAVAILABILITY

(75) Inventor: Roger A. Rawjewski, Lawrence, KS (US)

(73) Assignee: UNIVERSITY OF KANSAS, Lawrence, KS (US)

(21) Appl. No.: 13/263,838

(22) PCT Filed: Apr. 9, 2010
(86) PCT No.: PCT/US2010/030565

§ 371 Date: Apr. 9, 2012

Related U.S. Application Data
(60) Provisional application No. 61/168,014, filed on Apr. 9, 2009.

(51) Int. Cl. A61K 31/407; A61P 35/00

(52) U.S. Cl. 514/410

(57) ABSTRACT

A pharmaceutical composition for treating, inhibiting, or preventing cancer can include an indole-3-carbinol derivative compound in a pharmaceutically acceptable carrier that is configured for oral administration. The indole-3-carbinol derivative compound can have antitumor activity, and oral administration can provide blood bioavailability of about 0.5% to about 25%. The pharmaceutically acceptable carrier can include a hydroxyl-fatty acid PEG monoester and/or diester. The carrier can be a hydroxyl-fatty acid PEG ester that includes 12-hydroxy stearate. The carrier can be a hydroxyl-fatty acid PEG ester that includes a PEG having from about 100 MW to about 200,000 MW. The indole-3-carbinol derivative can be 2,10-dicarbethoxy-6-methoxy-5,7-dihydro-indolo-(2,3-b)carbazole.

### CROSS-REFERENCE

This patent application claims the benefit of U.S. Provisional Application No. 61/168,014, filed on Apr. 9, 2009, which provisional application is incorporated herein by specific reference in its entirety.

This invention was made with government support under N01-CN 35000 awarded by the National Cancer Institute. The government has certain rights in the invention.

### BACKGROUND

An indole-3-carbinol derivative has been found to be useful as a potential antitumor agent. The indole-3-carbinol derivative can be SR13668 (2,10-dicarbethoxy-6-methoxy-5,7-dihydro-indolo-(2,3-b)carbazole), or other derivatives thereof. However, the indole-3-carbinol derivative compounds have had limited success in being formulated sufficiently for use as a therapeutic. Additional information regarding the indole-3-carbinol derivative compounds can be found in U.S. Pat. No. 7,429,610, which is incorporated herein by specific reference in its entirety.

### SUMMARY

Generally, a pharmaceutical composition for treating, inhibiting, or preventing cancer can include an indole-3-carbinol derivative compound in a pharmaceutically acceptable carrier that is configured for oral administration. The indole-3-carbinol derivative compound can have antitumor activity, and oral administration can provide blood bioavailability of about 0.5% to about 25%. The pharmaceutically acceptable carrier can include a hydroxyl-fatty acid PEG monoester and/or diester. The carrier can be a hydroxyl-fatty acid PEG ester that includes 12-hydroxy stearate. The carrier can be a hydroxyl-fatty acid PEG ester that includes a PEG having from about 100 MW to about 200,000 MW. The indole-3-carbinol derivative can be 2,10-dicarbethoxy-6-methoxy-5,7-dihydro-indolo-(2,3-b)carbazole. The indole-3-carbinol derivative can be present from about 0.5 mg to about 15 mg per gram of pharmaceutically acceptable carrier. The indole-3-carbinol derivative can be 2,10-dicarbethoxy-6-methoxy-5,7-dihydro-indolo-(2,3-b)carbazole and present up to about 13 mg per gram of pharmaceutically acceptable carrier. The pharmaceutically acceptable carrier may also include free PEG up to about 50%. The composition can be configured as a dose that contains from about 10 mg to about 100 mg of the indole-3-carbinol derivative. The composition is a dose in the form of a gel capsule.

In one embodiment, the present invention can include a method of manufacturing a pharmaceutical composition as described herein. The method can include: obtaining powdered and/or crystalline indole-3-carbinol derivative; and combining the crystalline indole-3-carbinol derivative with the pharmaceutically acceptable carrier under heat and stirring to form a mixture. The method can also include grinding crystalline indole-3-carbinol derivative into a powder. The method can also include heating the mixture to at least about 65° C. The method can also include heating the mixture to less than about 110° C. For example, the mixture can be heated to between about 65° C. to about 95° C. The mixture can be configured into an oral formulation having the bioavailability. A capsule can be filled with the mixture to prepare a dose.

In one embodiment, the present invention can include a method of treating, inhibiting, and/or preventing cancer. The method can include: orally administering a pharmaceutical composition as described herein to a subject. The subject can have or can be susceptible to cancer. The subject may have been diagnosed with cancer. The treatment can include administering one or more doses of the composition one or more times daily. The treatment can include administering a therapeutically effective amount of the composition in order to treat, inhibit, and/or prevent cancer.

### BRIEF DESCRIPTION OF THE FIGURES

FIG. 1A is a chemical structure of SR13668.

FIG. 1B is a chemical structure of the hydroxyl-fatty acid PEG monoester and di-ester.

FIG. 2 is a pharmacokinetic profile of SR13668 following i.v. dosing in fed and oral gavage dosing in fed and fasted dogs. Data are presented for a single dose i.v. and seventh daily oral dose at 93.6 mg/m² (4.7 mg/kg) in DMSO:PEG300 (15:85, v/v) and Solutol®, respectively.

FIG. 3 is a pharmacokinetic profile of SR13668 following i.v. dosing in fed and oral dosing in monkeys. Data are presented for a single dose i.v. and seventh daily oral gavage dose at 84.2 mg/m² (7.0 mg/kg) in DMSO:PEG300 (15:85, v/v) and Solutol® or PEG400:Labrasol® (1:1, v/v), respectively.

FIG. 4 is a stability profile of SR13668 in Solutol stored as solid samples in Eppendorf tubes under two storage conditions.

FIG. 5 is a stability profile of SR13668/Solutol stored as aqueous samples under two storage conditions.

FIG. 6 is a stability profile of SR13668 in Solutol stored as solid samples under two storage conditions and combined with water before analysis

FIGS. 7A-C are dissolution profiles in water (FIG. 7A), SGF (FIG. 7B), and SIF (FIG. 7C).

### DETAILED DESCRIPTION

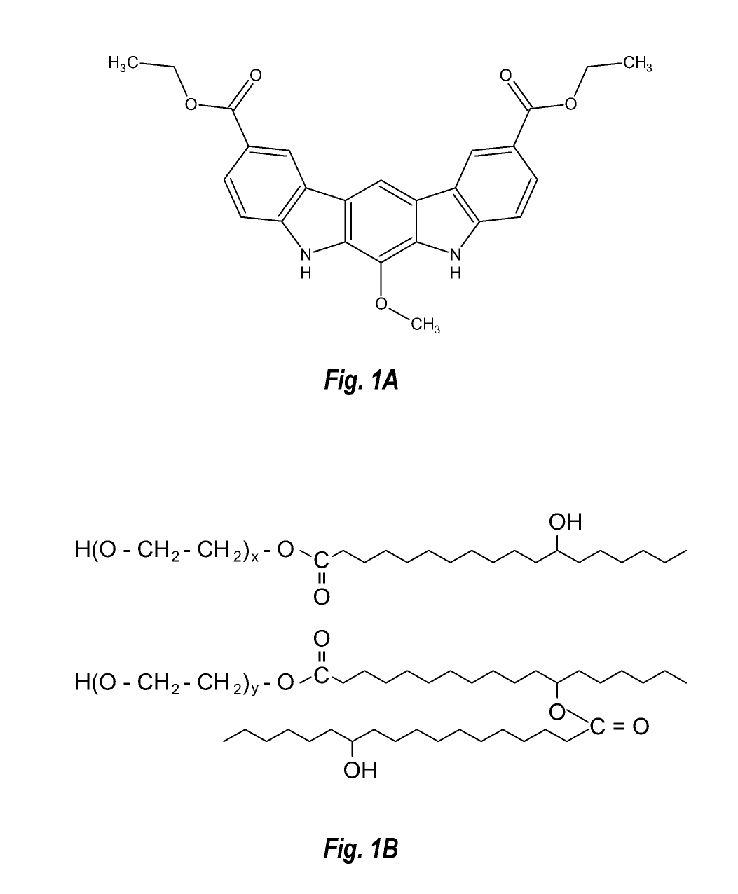

Generally, indole-3-carbinol derivative compounds may be used as antitumor agents or for other therapeutic uses. However, these indole-3-carbinol derivatives have limited bioavailability in current formulations. FIG. 1A shows the structure of an indole-3-carbinol derivative, SR13668 (2,10-dicarbethoxy-6-methoxy-5,7-dihydro-indolo-(2,3-b)carbazole), that has been shown to be a potential therapeutic for use as an antitumor agent, but that has not before now been successfully formulated for oral use with sufficient bioavailability. The SR13668 antitumor agent can now be formulated into an oral composition having increased bioavailability by being formulated with hydroxy-fatty acid polyethylene glycol esters, such as the commercially available Solutol as shown in FIG. 1B. The 2,10-dicarbethoxy-6-methoxy-5,7-dihydro-indolo-(2,3-b)carbazole compound had previously shown poor oral bioavailability in many formulations, and as such, the formulations recited herein have provided the surprising and unexpected results of sufficient bioavailability upon oral administration. While the SR13668 indole-3-carbinol derivative is a focus of the studies described herein, other indole-3-carbinol derivatives, such as the derivatives described in U.S. Pat. No. 7,429,610, may be similarly formulated as described herein.

Accordingly, the indole-3-carbinol derivatives, such as those shown in Formulas 1-4, can be formulated for oral administration and increased bioavailability for cancer therapy.

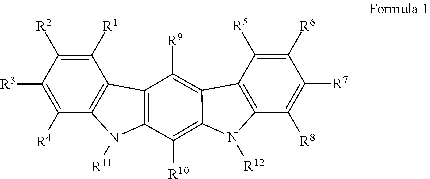

In Formula 1, the variables are defined as follows: R¹, R², R³, R⁴, R⁵, R⁶, R⁷, R⁸, R⁹, and R¹⁰ are substituents independently selected from the group of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano(—C≡N), isocyano (—N⁺≡C⁻), cyanato isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₆-C₂₀ arylamido (—NH—(CO)-aryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—S₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O—)), phospho (—PO₂), phosphino (—PH₂), and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms; and R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl, with the proviso that at least one of R¹, R², R³, R⁴, R⁵, R⁶, R⁷, R⁸, R⁹, R¹⁰, R¹¹, and R¹² is other than hydrogen. Exemplary compounds within the aforementioned group are those wherein R¹ through R¹² are as defined with the proviso that when R¹, R², R³, R⁴, R⁵, R⁶, R⁷, and R⁸ are selected from hydrogen, halo, alkyl, and alkoxy, then R¹¹ and R¹² are other than hydrogen and alkyl. Specific examples include: 5-Carbethoxy-6-ethoxycarbonyloxy-7H-indolo[2,3-b]carbazole; 6-Ethoxycarbonyloxy-5,7-dihydro-indolo[2,3-b]carbazole; 6-Methyl-5,7-dihydro-indolo[2,3-b]carbazole; 2,10-Dicarbethoxy-6-ethoxycarbonyloxy-5,7-dihydro-indolo[2,3-b]carbazole; 2,10-Dibromo-6-ethoxycarbonyloxy-5,7-dihydro-indolo[2,3-b]carbazole; 2,10-Dicarbethoxy-6-methyl-5,7-dihydro-indolo[2,3-b]carbazole; 2,10-Dicarbethoxy-6-(heptafluoropropyl)-5,7-dihydro-indolo[2,3-b]carbazole; 2,10-Dicarbethoxy-6-methoxy-5,7-dihydro-indolo[2,3-b]carbazole; 2,10-Dicarbethoxy-6-ethoxy-5,7-dihydro-indolo[2,3-b]carbazole; 2,10-Dicarbethoxy-6-(trifluoromethyl)-5,7-dihydro-indolo[2,3-b]carbazole; 2,10-Dicarbethoxy-6-(pentafluoroethyl)-5,7-dihydro-indolo[2,3-b]carbazole; 2,10-Dicarbethoxy-6-(n-propyl)-5,7-dihydro-indolo[2,3-b]carbazole; 2,10-Dicarbethoxy-6-(1,1,1-trifluoroethyl)-5,7-dihydro-indolo[2,3-b]carbazole; 2,6,10-tricarbethoxy-5,7-dihydro-indolo[2,3-b]carbazole; 2,10-Dicarbethoxy-6-ethoxycarbonyloxy-5,7-dimethyl-5,7-dihydro-indolo[2,3-b]carbazole; 6-Methoxy-5,7-dihydro-indolo[2,3-b]carbazole; 6-Ethoxy-5,7-dihydro-indolo[2,3-b]carbazole; 6-Methyl-5,7-dihydro-indolo[2,3-b]carbazole; 6-(Trifluoromethyl)-5,7-dihydro-indolo[2,3-b]carbazole; 6-(Pentafluoroethyl)-5,7-dihydro-indolo[2,3-b]carbazole; 6-(n-Propyl)-5,7-dihydro-indolo[2,3-b]carbazole; 5,7-Dimethyl-5,7-dihydro-indolo[2,3-b]carbazole-6-carboxylic acid ethyl ester; 6-Ethoxycarbonyloxy-5,7-dimethyl-5,7-dihydro-indolo[2,3-b]carbazole; [2-(5,7-Dihydro-indolo[2,3-b]carbazol-6-yloxy)-ethyl]-dimethyl-amine; 6-(2-Dimethylamino-ethoxy)-5,7-dihydro-indolo[2,3-b]carbazole; 2,10-Dicarbethoxy-6-(2-Dimethylamino-ethoxy)-5,7-bis-(2-dimethylamino-ethyl)-5,7-dihydro-indolo[2,3-b]-carbazole; 2,10-Dibromo-5,7-dimethyl-5,7-dihydro-indolo[2,3-b]carbazole-6-carboxylic acid ethyl ester; 2,10-Dibromo-5,7-dihydro-indolo[2,3-b]carbazole-6-carboxylic acid ethyl ester; Carbonic acid 2,10-dibromo-5,7-dihydro-indolo[2,3-b]carbazol-6-yl ester ethyl ester; Carbonic acid 2,10-bis-dimethylcarbamoyl-5,7-dihydro-indolo[2,3-b]carbazole-6-yl ester ethyl ester; 6-Methoxy-5,7-dihydro-indolo[2,3-b]carbazole-2,10-dicarboxylic acid bis-dimethylamide; 5,7-Dihydro-indolo[2,3-b]carbazole-2,10-dicarboxylic acid bis-dimethylamide; 2,10-Bis-methanesulfinyl-5,7-dihydro-indolo[2,3-b]carbazole; 2,10-Bis-methylsulfanyl-5,7-dihydro-indolo[2,3-b]carbazole; and 2,10-Bis-methanesulfonyl-5,7-dihydro-indolo[2,3-b]carbazole.

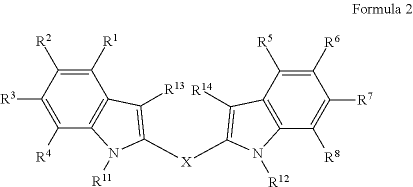

In Formula 2, the variables are defined as follows: R¹, R², R³, R⁴, R⁵, R⁶, R⁷, R⁸ R¹¹ and R¹² are as defined for Formula 1; R¹³ and R¹⁴ are defined as for R¹, R², R³, R⁴, R⁵, R⁶, R⁷, and R⁸, with the proviso that at least one of R¹³ and R¹⁴ is other than hydrogen; and X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹². Specific examples include: 3-Methylthio-2,2′-diindolylmethane; 3,3′-Dimethyl-2,2′-diindolylmethane; 3,3′-Dimethyl-5,5′-dicarbethoxy-2,2′-diindolylmethane; 3,3′-Dimethyl-5-carbethoxy-2,2′-diindolylmethane-5,5′-Dicarbethoxy-2,2′-diindolylmethane; N,N′-Dimethyl-3,3′-dimethyl-2,2′-diindolylmethane; N,N′-Dimethyl-3,3′-dimethyl-5,5′-dicarbethoxy-2,2′-diindolylmethane; N-Methyl-3,3′-dimethyl-5,5′-dicarbethoxy-2,2-diindolylmethane; N,N′-Dicarbethoxy-3,3′-dimethyl-5,5′-dicarbethoxy-2,2-diindolylmethane; and N-Carbethoxy-3,3′-dimethyl-5,5′-dicarbethoxy-2,2′-diindolylmethane.

Exemplary compounds within the aforementioned group are those wherein only one but not both of R² and R⁶ is amino, mono-substituted amino, or di-substituted amino.

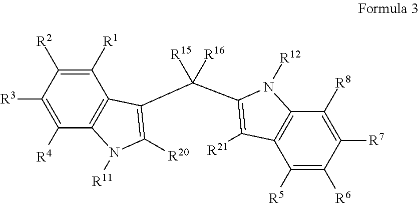

In Formula 3, the variables are defined as follows: R¹, R², R³, R⁴, R⁵, R⁶, R⁷, R⁸, R¹¹, R¹², and X are as defined for compounds having the structure of Formula (2); and R²⁰ and R²¹ are defined as for R¹, R², R³, R⁴, R⁵, R⁶, R⁷, and R⁸, Specific examples can include: 2,3′-Diindolylmethane; 2,3′-Dimethyl-5,5′-dicarbethoxy-2′,3-diindolylmethane; 2,3′-Dimethyl-2′,3-diindolylmethane; 5,5′-Dicarbethoxy-2′,3-diindolylmethane; 5-Carbethoxy-2,3′-dimethyl-2′,3-diindolylmethane; N,N′-Dimethyl-2,3′-diindolylmethane; N,N′-Dimethyl-2,3′-dimethyl-2′,3-diindolylmethane; N,N′-Dimethyl-2,3′-Dimethyl-5,5′-dicarbethoxy-2′,3-diindolylmethane; N-Methyl-2,3′-Dimethyl-5,5′-dicarbethoxy-2′,3-diindolylmethane; N,N′-Dicarbethoxy-2,3′-Dimethyl-5,5′-dicarbethoxy-2′,3-diindolylmethane; and N-Carbethoxy-2,3′-Dimethyl-5,5′-dicarbethoxy-2′,3-diindolylmethane.

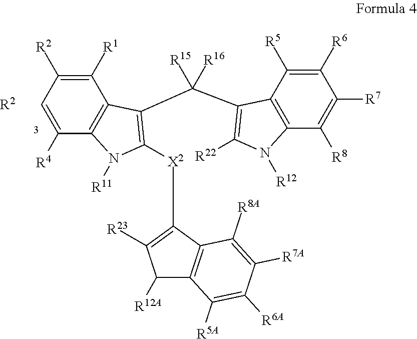

In Formula 4, the variables are defined as follows: R¹, R², R³, R⁴, R⁵, R⁶, R⁷, R⁸, R¹¹, R¹², and X are as defined for compounds having the structure of Formula (2); R^{5A}, R^{6A}, R^{7A}, R^{8A}, and R^{12A} are defined as for R⁵, R⁶, R⁷, R⁸, and R¹², respectively; R²² and R²³ are defined as for R²⁰ and R²¹ in the structure of Formula (3); and X¹ and X² are independently selected from O, S, arylene, heteroarylene, CR¹⁵R¹⁶ and NR¹⁷, or together form ═CR¹⁸R¹⁹ wherein R¹⁵, R¹⁶, R¹⁷, R¹⁸, and R¹⁹ are as defined previously with respect to compounds of Formula (2), with the proviso that at least one of R¹, R², R³, R⁴, R⁵, R⁶, R⁷, R⁸, R^{5A}, R^{6A}, R^{7A}, R^{8A}, R¹¹, R¹², R²² and R²³ is other than hydrogen. Specific examples can include: 2-(2-Carbethoxy-indol-3-ylmethyl)-2′-carbethoxy-3,3′-diindolylmethane; 2-(5-Bromo-indol-3-ylmethyl)-5,5′-dibromo-3,3-diindolylmethane; and 2-(5-Carbethoxy-indol-3-ylmethyl)-5,5′-dicarbethoxy-3,3′-diindolylmethane.

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, preferably 1 to about 12 carbon atoms. The term “lower alkyl” intends an alkyl group of 1 to 6 carbon atoms. Preferred substituents identified as “C₁-C₆ alkyl” or “lower alkyl” contain 1 to 3 carbon atoms, and particularly preferred such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The terms “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 18 carbon atoms, preferably 2 to 12 carbon atoms. The term “lower alkenyl” intends an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, preferably 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Preferred substituents identified as “C₁-C₆ alkoxy” or “lower alkoxy” herein contain 1 to 3 carbon atoms, and particularly preferred such substituents contain 1 or 2 carbon atoms (i.e., methoxy and ethoxy).

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 20 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 20 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Preferred aralkyl groups contain 6 to 24 carbon atoms, and particularly preferred aralkyl groups contain 6 to 16 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like.

The term “cyclic” refers to alicyclic or aromatic substituents that may or may not be substituted and/or heteroatom containing, and that may be monocyclic, bicyclic, or polycyclic.

The terms “halo” and “halogen” are used in the conventional sense to refer to a chloro, bromo, and fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

“Hydrocarbyl” refers to univalent hydrocarbyl radicals containing 1 to about 30 carbon atoms, preferably 1 to about 24 carbon atoms, more preferably 1 to about 18 carbon atoms, most preferably about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents.

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, heteroatom-containing alkenyl, and heteroatom-containing aryl.”

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. For example, treatment of a patient by administration of an anti-cancer agent of the invention encompasses chemoprevention in a patient susceptible to developing cancer (e.g., at a higher risk, as a result of genetic predisposition, environmental factors, or the like) and/or in cancer survivors at risk of cancer recurrence, as well as treatment of a cancer patient dual by inhibiting or causing regression of a disorder or disease.

By the terms “effective amount” and “therapeutically effective amount” of a compound of the invention is meant a nontoxic but sufficient amount of the drug or agent to provide the desired effect.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree.

A compound of the invention may be administered in the form of a salt, ester, amide, prodrug, active metabolite, analog, or the like, provided that the salt, ester, amide, prodrug, active metabolite or analog is pharmaceutically acceptable and pharmacologically active in the present context. Salts, esters, amides, prodrugs, active metabolites, analogs, and other derivatives of the active agents may be prepared using standard procedures known to those skilled in the art of synthetic organic chemistry and described, for example, by J. March, Advanced Organic Chemistry. Reactions, Mechanisms and Structure, 4th Ed. (New York: Wiley-Interscience, 1992).

For example, acid addition salts may be prepared from a free base (e.g., a compound containing a primary amino group) using conventional methodology involving reaction of the free base with an acid Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of any acidic moieties that may be present may be carried out in a similar manner using a pharmaceutically acceptable base such as sodium hydroxide, potassium hydroxide, ammonium hydroxide, calcium hydroxide, trimethylamine, or the like. Preparation of esters involves reaction of a hydroxyl group with an esterification reagent such as an acid chloride. Amides may be prepared from esters, using suitable amine reactants, or they may be prepared from an anhydride or an acid chloride by reaction with ammonia or a lower alkyl amine. Prodrugs, conjugates, and active metabolites may also be prepared using techniques known to those skilled in the art or described in the pertinent literature. Prodrugs and conjugates are typically prepared by covalent attachment of a moiety that results in a compound that is therapeutically inactive until modified by an individual's metabolic system.

In addition, those novel compounds containing chiral centers can be in the form of a single enantiomer or as a racemic mixture of enantiomers. In some cases, i.e., with regard to certain specific compounds illustrated herein, chirality (i.e., relative stereochemistry) is indicated. In other cases, it is not, and such structures are intended to encompass both the enantiomerically pure form of the compound shown as well as a racemic mixture of enantiomers. Preparation of compounds in enantiomerically form may be carried out using an enantioselective synthesis; alternatively, the enantiomers of a chiral compound obtained in the form of the racemate may be separated post-synthesis, using routine methodology.

Other derivatives and analogs of the active agents may be prepared using standard techniques known to those skilled in the art of synthetic organic chemistry, or may be deduced by reference to the pertinent literature.

The compound can now be formulated with hydroxy-fatty acid polyethylene glycol monoesters or di-esters in order to prepare pharmaceutical preparations for oral administration. The hydroxyl-fatty acid polyethylene glycol esters can be prepared by conjugating a PEG polymer to a carboxylic acid group of a hydroxyl-fatty acid or hydroxyl-fatty acid ester to form an ester. The hydroxy-fatty acid esters can include any hydroxyl-fatty acid component, such as a C₄-C₂₄ hydroxyalkyl with the hydroxyl group being at any location. For example, the hydroxyl-fatty acid can be hydroxystearate, with 12-hydroxy stearate being an example.

The PEG can be of any molecular weight, such as between about 100 MW to about 200,000 MW, between about 500 MW to about 100,000 MW, between about 750 MW to about 50,000 MW, between about 1,000 and about 40,000 MW, between about 3,000 to about 27,000 MW, or about 5,000 to about 26,000 MW.

A specific example of a hydroxy-fatty acid polyethylene glycol monoester and/or di-ester can include Solutol, which can include a mixture of polyethylene glycol mono- and di-esters of 12-hydroxystearic acid with about 30% of free polyethylene glycol. Solutol can also be referred to as polyethylene glycol 660 12-hydroxystearate and Macrogol 15 Hydroxystearate.

Multiple formulations of SR13668 have been developed to increase the systemic concentration or exposure to SR13668 following oral administration. An improved composition can include Solutol, which has hydroxy-fatty acid polyethylene glycol esters. The Solutol compositions were an improvement over PEG400:Labrasol formulations, which is surprising and unexpected. PEG400:Labrasol includes PEG:Caprylocaproyl Macrogolglycerides (PEG:Polyoxylglycerides).

PEG400:Labrasol exhibited a very poor oral bioavailability (<1%) in both rats and dogs. Therefore, a study was initiated to develop and evaluate in dogs and non-human primates formulations with a more favorable oral bioavailability. Two formulations utilizing surfactant/emulsifiers, PEG400:Labrasol® and Solutol, were tested. The Solutol® formulation yielded better bioavailability reaching a maximum of about 14.6% and 7.3% in dogs and monkeys, respectively, following nominal oral dose of ca. 90 mg SR13668/m². Blood levels of SR13668 were consistently about 3 fold higher than those in plasma in both species. SR13668 did not cause untoward hematology, clinical chemistry, or coagulation effects in dogs or monkeys with the exception of a modest, reversible increase in liver function enzymes in monkeys.

The increase in oral bioavailability of SR13668 observed with Solutol formulations are surprising and unexpected in view of the extremely low aqueous solubility of SR13668, and thereby, offer the potential of SR13668 as well as other indole-3-carbinol derivatives to be useful in therapies, such as the treatment, inhibition, or prevention of cancer.

Under the conditions tested, the highest bioavailability achieved was about 14% using Solutol® as a vehicle in dogs. Similarity in % F and % F_{abs} values suggests that the low bioavailability of SR13668 is mainly due to its low absorption. Presystemic clearance doesn't appear to play a major role which is consistent with slow metabolism of SR13668 in rat microsomes (Green C., unpublished results). Dogs exhibited a two-fold higher bioavailability than monkeys with comparable doses in Solutol®. SR13668 tended to have higher bioavailability in Solutol® than in PEG400:Labrasol®. Fed or fasted condition did not have an effect of bioavailability. SR13668 tended to concentrate in blood cells with a whole blood: plasma concentration range almost 3 in all cases. Bioavailability estimates were similar between whole blood and plasma. Increasing the dose fourfold in PEG400:Labrasol® resulted in about a fourfold decrease in the bioavailability of SR13668 in monkeys. Reasons for this decrease are not clear but it is conceivable that SR13668 may have come out of suspension upon administration and precipitated in the gastrointestinal tract.

### Examples

### Test Article and Formulation Vehicles

SR13668 was provided to the Division of Cancer Prevention, National Cancer Institute by ScinoPharm, Taiwan, with a Certificate of Analysis confirming identity by

NMR, IR and MS and reported purity of 99% by HPLC. The formulation vehicles PEG 300, PEG 400, and DMSO (dimethyl sulfoxide) were purchased from Sigma Aldrich Chemical Co. (St. Louis, Mo.); Solutol® HS15 was obtained from BASF (Florham Park, N.J.); and Labrasol® was purchased from Gattefosse USA (Paramus, N.J.).

### Animals

Four non-naïve male beagle dogs (approximately 3 years of age; Ridglan Farms Inc. Mt. Horeb, Wis.) and four non-naïve male cynomolgus monkeys [cynos (macaca fasicularis); approximately 7 to 8 years of age; Charles River Laboratories, Inc., Houston, Tex.] were used in this study. Prior to experimental initiation for the present study, the attending veterinarian certified that the animals were healthy and free from disease and parasites.

### Study Design and Dosage

For both species, each animal was part of each experimental group, with a 7-day interval between treatments to allow for washout of the dosing formulation and its effects. Dosing formulations of SR13668 were administered by oral gavage (intragastric) or intravenous (i.v.) injection as a single dose or once daily for seven consecutive days. Oral to doses were administered at a dosing volume of 5 mL/kg of body weight, while intravenous doses were administered at a dosing volume of 0.5 mL/kg of body weight. Dogs were dosed with 93.6 mg/m² SR13668 intravenously in DMSO:PEG300 15:85 (v/v) or orally in Solutol® vehicle (two groups, fed and after an overnight fast). Monkeys were dosed with 84.2 mg/m² SR13668 intravenously in DMSO:PEG300 15:85 (v/v) or orally (336.7 mg/m² SR13668 for the oral high dose group) in PEG400:Labrasol® 1:1 (v/v) or Solutol® vehicles. Vehicle control groups were used to assess the tolerability of the vehicle and for clinical pathology evaluations.

Animals were observed at hours 0.5 (dogs)/1 (monkeys) and 4 hours post-dose on dosing days, as well as daily during washout periods, for any unusual behavioral activity, observable changes in appearance, and/or adverse clinical signs. At the end of each dosing period, the animals were examined for detailed clinical signs and symptoms (i.e., alterations of teeth, nose, eyes, perineum, pelage and body orifices; changes in appearance or behavior) and/or presence of any tissue masses. Body weights were recorded prior to treatment and daily during each testing interval. Quantitative (grams/day) food consumption data were recorded for dogs on a daily basis during each testing interval. Qualitative food consumption observations were recorded for monkeys on a daily basis during each testing interval. Blood samples for clinical pathology (clinical chemistry, hematology, and coagulation) parameter evaluation were collected from dogs and monkeys prior to dosing and on Days 2 and 8 post-dose, as well as from monkeys on Days 15 and 30 post-dose.

### Blood Collection for Analysis of SR13668

Blood samples were collected from each animal at 10 time points (0, 0.25, 0.5, 1, 2, 4, 6, 8, 12 and 24 hours post-dose) during the first and last 24-hour segment of each dosing regimen. For single intravenous dose experiments, samples were collected during the 24-hour post-dose period only. Samples were transferred to Vacutainer tubes containing ethylenediaminetetraacetic acid (EDTA; Fisher Scientific, Pittsburgh, Pa.). The tubes were inverted several times to mix and then placed on ice until storage or centrifugation for plasma preparation. After centrifugation, the plasma was transferred into storage tubes (0.5 mL), which were placed on dry ice and then stored frozen (approximately −70° C.).

### Analytical Method

Levels of SR13668 in plasma and blood were measured using a tandem mass to spectrometer (API 3000; Applied Biosystems/MDS Sciex, Foster City, Calif.) equipped with a high performance liquid chromatograph (Agilent 1200; Agilent Technologies, Wilmington, Del.). For SR13668 determination, a 100 μL blood or plasma aliquot was mixed with 1 mL of acetonitrile (ACN; Sigma-Aldrich, St. Louis, Mo.). After vortex-mixing for one minute, the sample was centrifuged at 4° C. and 7000 RPM for 10 minutes to remove precipitated proteins, and the supernatant was transferred to a clean tube and dried under nitrogen flow at room temperature (approximately 25° C.). After the evaporation was completed, the residue was reconstituted in 500 μL of ACN/water (v/v 70:30), and vortex-mixed and centrifuged again. An aliquot of the resulting supernatant was transferred to an autosampler tube for instrumental analysis.

A freshly prepared SR13668 standard curve was analyzed along with samples on each day of analysis. The chromatographic column was a Luna 3μ C18(2) 110 Å 30×2.0 mm (Phenomenex, Torrance, Calif.). The column temperature was maintained at 25° C., and a flow rate of 0.30 mL/min was used. The mobile phase consisted of MPA: formic acid in water (0.05%, v/v) and MPB: formic acid in ACN (0.05%, v/v). The mobile phase gradient was as follows: after injection, initial conditions with MPA at 40% were held for 0.01 min, decreased to 5% and held constant for 3 min, returning to initial conditions for another 3 min of re-equilibration time. Retention time of SR13668 was approximately 2.4 min. Total run time was 6 min. A turbo ion spray interface was used as the ion source operating in negative ion mode. Acquisition was performed in multiple reaction monitoring mode using ions 429.15 (Q1) and 414.12 (Q3) Dalton. Ion spray voltage was −4200 V, ion source temperature was 340° C., and collision energy was −30 V.

### Pharmacokinetic and Statistical Analysis

Pharmacokinetic (PK) analysis was performed on plasma and whole blood SR13668 concentration data on an individual animal basis using WinNonlin Professional Edition version 4.1 (Pharsight Inc., Mountain View, Calif.). The noncompartmental model for extravascular input was used for all PK analyses for oral (intragastric gavage) administration groups. The noncompartmental model for i.v.-bolus input was used for all PK analyses for i.v. administration groups. Area under the plasma concentration-time curve (AUC) from time zero to the last measured concentration was estimated by the linear trapezoidal rule up to Cₘₐₓ (maximum observed plasma concentration), followed by the log trapezoidal rule for the remainder of the curve. Area under the plasma concentration-time curve extrapolated to infinity is defined as AUC_{0-∞}=AUC₀₋ₜ+Cₜ/λ_{z}, where λ_{z} is the disposition rate constant estimated using log-linear regression during the terminal elimination phase and Cₜ is the last measureable plasma concentration. Statistical analyses were performed for t_{1/2}(elimination half-life), Tₘₐₓ (time of occurrence of maximum plasma concentration), Cₘₐₓ, AUC_{0-∞}, Vz/F (apparent volume of distribution), CL/F (apparent total body clearance), MRT (mean residence time) and F systemic availability of the administered dose) using log-transformed PK parameter data (with the exception of t_{1/2} and Tₘₐₓ). For maximum observed concentration (Cₘₐₓ) and area under the concentration-time curve (AUC), the data were normalized to the body surface area dose (i.e., mg SR13668/m²) prior to log-transformation. Systat software (Systat Software Inc., Chicago, Ill.; version 10.2) was used to analyze pharmacokinetic parameter data via repeated measure design and using general linear model computations to test changes across the repeated measures (within subjects) as well as differences between groups of subjects (between subjects). For each pharmacokinetic parameter, the tests were performed either by paired t-tests or repeated measure analysis followed, as necessary, by the post hoc Tukey's test (p≦0.05).

Animals were observed at least at 0.5 and 4 hours post-dosing and daily for any unusual behavioral activity, observable changes in appearance, and clinical signs. There were no mortalities or morbidity in the study. The only adverse treatment-related clinical observations included vomiting in fasted dogs and soft stools and diarrhea in orally but not i.v. dosed monkeys. No significant treatment-related effects on body weights, body weight gains or food consumption were seen in either the dogs or monkeys during this study.

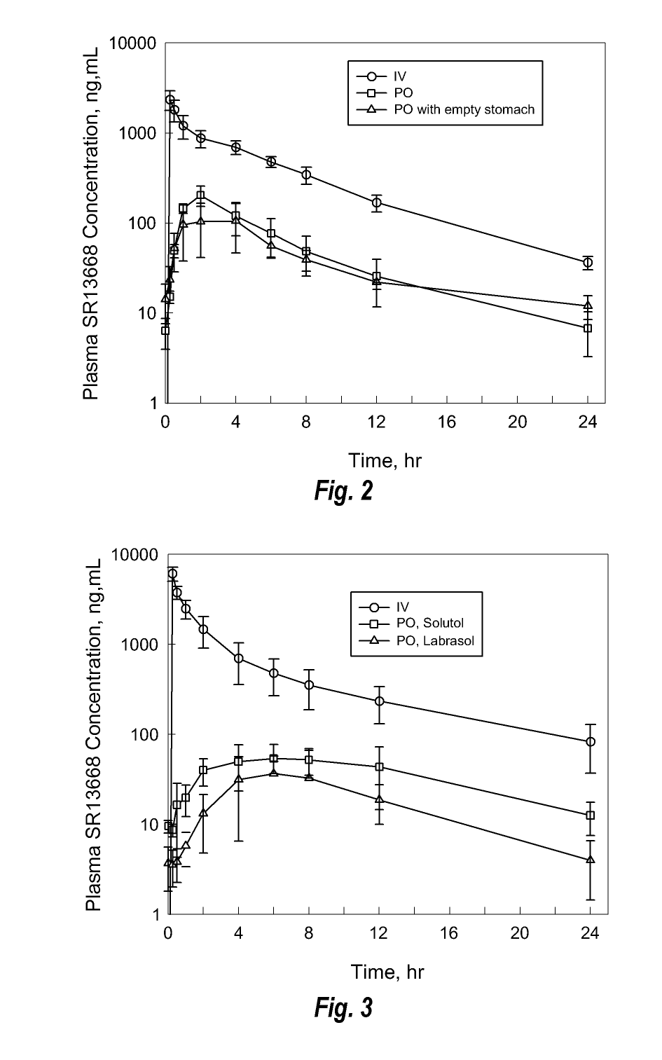

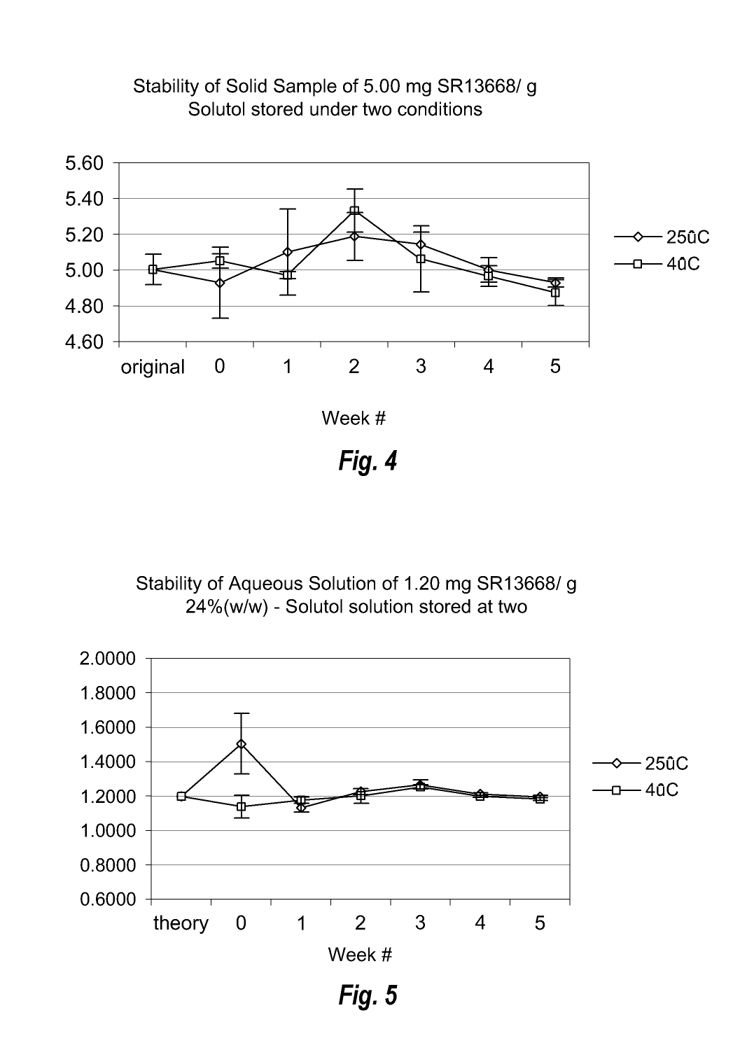

Plasma SR13668 concentration-time profiles following i.v. and oral gavage administration of SR13668 are presented for dogs and monkeys in FIGS. 2 and 3, respectively. Summaries of pharmacokinetic parameters in plasma and blood for both dogs and monkeys are presented in Tables 1A-1B and 2A-2B, respectively. The data are presented following the first day of i.v. dosing and the seventh oral dose of SR13668. The corresponding dog data for whole blood are presented in Tables 2A. Whole blood concentrations of SR13668 were consistently nearly 3-fold greater than those in plasma throughout the study in both species. The clearance was similar in dogs and monkeys following the i.v. dosing. Oral bioavailability tended to be slightly higher in whole blood as compared to plasma. Plasma and blood oral bioavailability ranged from 0.7 to 14.6% and 1.1 to 17.2%. Greater bioavailability following a comparable dose in the Solutol® vehicle based on a body surface area was observed in dogs than in monkeys, 14.6% vs. 7.3%. Solutol® yielded greater bioavailability in monkeys than PEG400:Labrasol® vehicle.

Increasing the dose four fold in PEG400:Labrasol® in monkeys resulted in a lower oral bioavailability. In order to distinguish low absorption from high first-pass presystemic clearance as the contributing factor for the low bioavailability, the fraction absorbed, F_{abs}, was estimated as F/(1-CL/Q) where Q represents hepatic blood flow in the respective species, and CL is the calculated blood clearance for SR13668 (Tables 3A-3B). Values for bioavailability (% F) were slightly lower but very close to the corresponding fraction absorbed (% F_{abs}) values.

TABLE 1A

| | DOGS | | |
|---|---|---|---|
| | 93.6 mg/m² | | |
| | 4.68 mg/kg | 93.6 mg/m² | 93.6 mg/m² |
| | i.v. | 4.68 mg/kg | 4.68 mg/kg |
| | DMSO:PEG300 | oral gavage | oral gavage |
| | 15:85, v/v | Solutol ® | Solutol ® |
| | Fed | Fed | Fasted |
| t_{1/2} (hr) | 5.0 ± 0.2^{g} | 5.6 ± 1.4 | 9.7 ± 2.4 |
| Tₘₐₓ (hr) | 0.25^{a} | 1.8 ± 0.5^{a} | 2.8 ± 1.5 |
| Cₘₐₓ (ng/mL) | 2356 ± 573^{a,g} | 207 ± 48^{a} | 113 ± 58 |
| AUC_{0-∞} | 8395 ± 1193^{a} | 1237 ± 414^{a} | 1100 ± 339 |
| hr * ng/mL | | | |
| V_{Z}/F (L/kg) | 4.05 ± 0.54^{a} | 32.4 ± 9.6^{a} | 64.0 ± 24 |
| CL/F (L/hr/kg) | 0.566 ± 0.081^{a} | 4.18 ± 1.6^{a} | 4.58 ± 1.4 |
| MRT (hr) | 6.3 ± 0.6 | 7.1 ± 1.1 | 12.8 ± 4.2 |
| F_{0-∞} (%) | 100 | 14.6 ± 3.9¹ | 13.3 ± 4.0 |

TABLE 1B

| | MONKEYS | | | |
|---|---|---|---|---|
| | 84.2 mg/m² | 84.2 mg/m² | 336.7 mg/m² | |
| | 7.02 mg/kg | 7.02 mg/kg | 28.06 mg/kg | 84.2 mg/m² |
| | i.v. | oral gavage | oral gavage | 7.02 mg/kg |
| | DMSO:PEG300 | PEG400:Labrasol ® | PEG400:Labrasol ® | oral gavage |
| | 15:85, v/v | 1:1 (v/v) | 1:1 (v/v) | Solutol ® |
| | Fed | Fed | Fed | Fed |
| t_{1/2} (hr) | 7.6 ± 0.8^{g} | 6.0 ± 1.3 | 5.0 ± 1.2 | 7.4 ± 0.7 |
| Tₘₐₓ (hr) | 0.25^{c,d} | 5.0 ± 1.2^{c} | 3.5 ± 1.9 | 8.0 ± 3.3^{c} |
| Cₘₐₓ (ng/mL) | 6094 ± 1080^{c,d,g} | 40.5 ± 38^{c} | 43.7 ± 9.2 | 71.4 ± 16^{d} |
| AUC_{0-∞} | 13334 ± 4925^{c,d} | 437 ± 312^{c,i} | 367 ± 154ⁱ | 837 ± 144^{d} |
| hr * ng/mL | | | | |
| V_{Z}/F (L/kg) | 6.36 ± 2.2^{c,d} | 203 ± 130^{c} | 603 ± 236 | 90.5 ± 9.9^{d} |
| CL/F (L/hr/kg) | 0.580 ± 0.20^{c,d} | 22.3 ± 12^{c,e,f} | 86.6 ± 32ⁱ | 8.55 ± 1.4^{d,e} |
| MRT (hr) | 6.9 ± 1.1^{c,d} | 11.6 ± 1.3^{c} | 9.1 ± 2.4 | 12.8 ± 2.1^{d} |
| F_{0-∞} (%) | 100 | 3.3 ± 2.0^{f} | 0.72 ± 0.28ⁱ | 7.3 ± 2.9ⁱ |

Values are presented as means ± SD.
Statistically significant difference between:
^{a}oral gavage, fed dogs and i.v. dogs for given parameter (excluding F)
^{b}oral gavage, fed dogs and oral gavage, fasted dogs for given parameter
^{c}oral gavage, low dose (84.2 mg/m²) monkeys (Labrasol ®) and i.v. monkeys for given parameter (excluding F)
^{d}oral gavage, low dose (84.2 mg/m²) monkeys (Solutol ®) and i.v. monkeys for given parameter (excluding F)
^{e}oral gavage, low dose (84.2 mg/m²) monkeys (Solutol ®) and oral gavage, low dose (84.2 mg/m²) monkeys (Labrasol ®) for given parameter
^{f}oral gavage, high dose (336.7 mg/m²) monkeys (Labrasol ®) and oral gavage, low dose (84.2 mg/m²) monkeys (Labrasol ®) for given parameter
^{g}i.v. monkeys and i.v. dogs for given parameter (excluding F)
ⁱoral gavage, low dose (84.2 mg/m²) monkeys (Solutol ®) and oral gavage, fed dogs (Solutol ®)

TABLE 2A

| | DOGS | | |
|---|---|---|---|
| | 93.6 mg/m² | | |
| | 4.68 mg/kg | 93.6 mg/m² | 93.6 mg/m² |
| | iv | 4.68 mg/kg | 4.68 mg/kg |
| | DMSO:PEG300 | oral gavage | oral gavage |
| | 15:85, v/v | Solutol ® | Solutol ® |
| | Fed | Fed | Fasted |
| t_{1/2} (hr) | 5.0 ± 0.1^{g} | 5.9 ± 1.5 | 7.0 ± 1.8^{h} |
| Tₘₐₓ (hr) | 0.25^{a} | 1.8 ± 0.5^{a} | 2.8 ± 1.5 |
| Cₘₐₓ (ng/mL) | 5217 ± 742^{a,g,h} | 575 ± 152^{a,h} | 277 ± 87^{h} |
| AUC_{0-∞} | 19509 ± 3158^{a,g,h} | 3414 ± 1286^{a,h} | 2681 ± 577^{h} |
| hr * ng/mL | | | |
| V_{Z}/F (L/kg) | 1.76 ± 0.23^{a,h} | 12.8 ± 4.8^{a,h} | 18.6 ± 7.5^{h} |
| CL/F (L/hr/kg) | 0.244 ± 0.034^{a,h} | 1.54 ± 0.62^{a,h} | 1.80 ± 0.36^{h} |
| MRT (hr) | 6.7 ± 0.6 | 7.2 ± 0.9 | 10.9 ± 3.1^{h} |
| F_{0-∞} (%) | 100 | 17.2 ± 4.3^{h,i} | 14.2 ± 4.5 |

TABLE 2B

| | MONKEYS | | | |
|---|---|---|---|---|
| | 84.2 mg/m² | 84.2 mg/m² | 336.7 mg/m² | |
| | 7.02 mg/kg | 7.02 mg/kg | 28.06 mg/kg | 84.2 mg/m² |
| | iv | oral gavage | oral gavage | 7.02 mg/kg |
| | DMSO:PEG300 | PEG400:Labrasol ® | PEG400:Labrasol ® | oral gavage |
| | 15:85, v/v | 1:1 (v/v) | 1:1 (v/v) | Solutol ® |
| | Fed | Fed | Fed | Fed |
| t_{1/2} (hr) | 8.6 ± 1.7^{c,g} | 5.8 ± 0.9^{c} | 6.8 ± 1.2^{h} | 7.7 ± 0.6 |
| Tₘₐₓ (hr) | 0.25^{c,d} | 7.0 ± 0.35^{c} | 3.5 ± 1.9 | 6.5 ± 3.8^{h} |
| Cₘₐₓ (ng/mL) | 11090 ± 776^{c,d,g,h} | 129 ± 140^{c,h} | 168 ± 35^{h} | 227 ± 56^{d,h} |
| AUC_{0-∞} hr * ng/mL | 35429 ± 12438^{c,d,g,h} | 1564 ± 1332^{c,e,h} | 1523 ± 445^{h} | 2489 ± 377^{d,e,h} |
| V_{Z}/F (L/kg) | 2.63 ± 0.70^{c,d,h} | 61.4 ± 44^{c,h} | 195 ± 73^{h} | 31.6 ± 5.0^{d,h} |
| CL/F (L/hr/kg) | 0.218 ± 0.078^{c,d,h} | 7.25 ± 5.4^{c,e,h} | 19.9 ± 6.8^{h} | 2.86 ± 0.41^{d,e,h} |
| MRT (hr) | 9.0 ± 2.2^{h} | 11.5 ± 1.2 | 10.9 ± 2.2 | 13.3 ± 1.1 |
| F_{0-∞} (%) | 100 | 3.8 ± 3.1^{e,h} | 1.1 ± 0.44^{h} | 8.3 ± 3.1^{e,h,i} |

Values are presented as means ± SD.
Statistically significant difference between:
^{a}oral gavage, fed dogs and i.v. dogs for given parameter (excluding F)
^{b}oral gavage, fed dogs and oral gavage, fasted dogs for given parameter
^{c}oral gavage, low dose (84.2 mg/m²) monkeys (Labrasol ®) and i.v. monkeys for given parameter (excluding F)
^{d}oral gavage, low dose (84.2 mg/m²) monkeys (Solutol ®) and i.v. monkeys for given parameter (excluding F)
^{e}oral gavage, low dose (84.2 mg/m²) monkeys (Solutol ®) and oral gavage, low dose (84.2 mg/m²) monkeys (Labrasol ®) for given parameter
^{f}oral gavage, high dose (336.7 mg/m²) monkeys (Labrasol ®) and oral gavage, low dose (84.2 mg/m²) monkeys (Labrasol ®) for given parameter
^{g}i.v. monkeys and i.v. dogs for given parameter (excluding F)
^{h}blood and plasma for given group and
ⁱoral gavage, low dose (84.2 mg/m²) monkeys (Solutol ®) and oral gavage, fed dogs (Solutol ®) parameter

TABLE 3A

| | DOGS | | |
|---|---|---|---|
| | 93.6 mg/m² | | |
| | 4.68 mg/kg | 93.6 mg/m² | 93.6 mg/m² |
| | iv | 4.68 mg/kg | 4.68 mg/kg |
| | DMSO:PEG300 | oral gavage | oral gavage |
| | 15:85, v/v | Solutol ® | Solutol ® |
| | Fed | Fed | Fasted |
| CL (mL/min/kg) | 4.07 | 25.7 | 30.0 |
| Q Blood flow | 30.9 | 30.9 | 30.9 |
| (hepatic)¹¹ | | | |
| (mL/min/kg) | | | |
| ER CLᵢᵥ/Q | 0.14 | | |
| F (%) | 100 | 17.2 | 14.2 |
| F_{abs} (%) F/(1-ER) | | 20.0 | 16.5 |

TABLE 3B

| | MONKEYS | | | |
|---|---|---|---|---|
| | 84.2 mg/m² | 84.2 mg/m² | 336.7 mg/m² | 84.2 mg/m² |
| | 7.02 mg/kg | 7.02 mg/kg | 28.06 mg/kg | 7.02 mg/kg |
| | iv | oral gavage | oral gavage | oral |
| | DMSO:PEG300 | PEG400:Labrasol ® | PEG400:Labrasol ® | gavage |
| | 15:85, v/v | 1:1 (v/v) | 1:1 (v/v) | Solutol ® |
| CL | Fed | Fed | Fed | Fed |
| (mL/min/kg) | | | | |
| QBlood flow | 3.63 | 121 | 332 | 47.7 |
| (hepatic)¹¹ | | | | |
| (mL/min/kg) | | | | |
| ER CLᵢᵥ/Q | 43.6 | 43.6 | 43.6 | 43.6 |
| F (%) | 0.083 | | | |
| F_{abs} (%) | 100 | 3.8 | 1.1 | 8.3 |
| F/(1-ER) | | | | |
| | | 4.1 | 1.2 | 9.1 |

During the study, clinical pathology (clinical chemistry, hematology, and coagulation) parameters were also monitored. No treatment-related effects on any clinical pathology variables were observed for dogs. The same was true for the monkeys except for modest increases in liver enzymes, lactate dehydrogenase (LDH), alanine aminotransferase (ALT), and aspartate aminotransferase (AST) (Table 4). These increases appeared to be due to SR13668, since there were no increases in any of these enzymes in the two corresponding vehicle groups. These changes were reversible on discontinuation of dosing (Table 5).

TABLE 4

| | | | | | | ALT | AST | LDH |
|---|---|---|---|---|---|---|---|---|
| | | | | | | (IU/L) | (IU/L) | (IU/L) |
| DOGS | 0 | mg/m² | oral | Solutol ® | Fed | 42.3 ± 4.8 | 34.5 ± 6.8 | 84.8 ± 35 |
| | 0 | mg/kg | | | | | | |
| | 93.6 | mg/m² | iv | DMSO:PEG300 | Fed | 41.8 ± 5.4 | 30.8 ± 5.2 | 54.8 ± 23 ^{c} |
| | 4.68 | mg/kg | | 15:85, v/v | | | | |
| | 93.6 | mg/m² | oral | Solutol ® | Fed | 39.5 ± 4.7 | 29.3 ± 3.6 | 30.3 ± 6.2 ^{c} |
| | 4.68 | mg/kg | gavage | | | | | |
| | 93.6 | mg/m² | oral | Solutol ® | Fasted | 44.8 ± 10 | 30.8 ± 7.2 | 56.3 ± 9.4 |
| | 4.68 | mg/kg | gavage | | | | | |
| MONKEYS | 0 | mg/m² | oral | PEG400:Labrasol ® | Fed | 48.8 ± 13 | 46.0 ± 4.8 | 304 ± 66 |
| | 0 | mg/kg | | 1:1 (v/v) | | | | |
| | 84.2 | mg/m² | iv | DMSO:PEG300 | Fed | 88.5 ± 37 | 134 ± 41 ^{b} | 730 ± 210 ^{c} |
| | 7.02 | mg/kg | | 15:85, v/v | | | | |
| | 84.2 | mg/m² | oral | PEG400:Labrasol ® | Fed | 86.0 ± 24 | 138 ± 75 | 795 ± 455 |
| | 7.02 | mg/kg | gavage | 1:1 (v/v) | | | | |
| | 336.7 | mg/m² | oral | PEG400:Labrasol ® | Fed | 108 ± 28 ^{a} | 188 ± 34 ^{b} | 753 ± 242 ^{c} |
| | 28.06 | mg/kg | gavage | 1:1 (v/v) | | | | |
| | 84.2 | mg/m² | oral | Solutol ® | Fed | 95.5 ± 33 | 150 ± 40 ^{b} | 779 ± 403 |
| | 7.02 | mg/kg | gavage | | | | | |

Values are presented as means ± SD for n = 4 on day 2 following a single dose of SR13668.
^{a} ALT: statistically significant difference between treated and control (0 mg/m²) group, with each group per species compared separately to its control group
^{b} AST: statistically significant difference between treated and control (0 mg/m²) group, with each group per species compared separately to its control group
^{c} LDH: statistically significant difference between treated and control (0 mg/m²) group, with each group per species compared separately to its control group

TABLE 5

| | | ALT | AST | LDH |
|---|---|---|---|---|
| Monkeys | Day | (IU/L) | (IU/L) | (IU/L) |
| 84.2 mg/m² | 1 | 41.0 ± 10 | 25.5 ± 4.1 | 221 ± 75 |
| 7.02 mg/kg | (pre- | | | |
| oral gavage | dose) | | | |
| PEG400:Labrasol ® | 2 | 112 ± 52 | 234 ± 115 | 1022 ± 401 |
| 1:1 (v/v) | 8 | 100 ± 38 ^{a} | 178 ± 86 ^{b} | 1035 ± 596 |
| Fed | 15 | 41.3 ± 10 | 24.0 ± 2.9 | 184 ± 39 |
| | 30 | 33.3 ± 2.2 | 22.5 ± 4.3 | 168 ± 39 |
| Range of normal | | 16-80 | 39-80 | 313-1034 |
| historical values | | | | |
| in the testing | | | | |
| laboratory | | | | |
| Range of normal | | 20-60 | 25-60 | 300-600 |
| values¹⁵ | | | | |

Values are presented as means ± SD for n = 4 except for n = 2 on day 2.
Single daily dose of SR13668 was administered to monkeys on days 1 through 7. Liver enzymes were monitored 24 hr after a single dose on days 2 and 8 and after discontinuation of dosing, days 15 and 30.
^{a} ALT: statistically significant difference between given day and Day 1
^{b} AST: statistically significant difference between given day and Day 1
^{c} LDH: statistically significant difference between given day and Day 1

### Procedure for Making Human Dosage Form

Prepare 50 g of matrix (hydroxyl-fatty acid PEG ester) with 5 mg SR13668/g matrix at 65° C., stirring for 24-48 hrs using ground crystals of SR13668. The mixture is transfer to 60-mL heated syringe, and dispense into 4 batches of 8 “00” hard gel capsules while weighing. Final weight of 8 capsules will be 7.6 g to give a total dose of 38 mg SR13668 per person. Note a “00” capsule body holds approximately 1 g of the matrix material. The matrix is allowed to harden and the capsule is capped. These capsules can be used in regimens ranging from one to four capsules per dose, and up to 3 doses per day.

Table 6 provides a summary of compositions and the components thereof for various oral doses for a comparative analysis.

### Stability Study

Prepare a batch of 32 capsules as above testing preparation method. Transfer residual sample not used in capsules to a centrifuge tube kept at 65° C. Centrifuge for 2 min at max rpm. Assay top, middle and bottom samples to look for indications of sample inhomogeniety. Assay 3 capsules at t=0 using HPLC/fluorescence. Store the remaining capsules in HDPE screw cap bottles in stability oven at 25° C., 60% RH. Assay 3 capsules at 2 weeks and 4 weeks. Repeat for all formulations except Labrisol/PEG400. Adjust HPLC method from isocratic to gradient assay to look for any degradation products. It is also possible to run a high concentration sample under UV detection, (as well as the diluted Fluorescence sample) to look for degradants. Stability is shown in Table 7

TABLE 6

| | | | | | | | | Total | Total (g) |
|---|---|---|---|---|---|---|---|---|---|
| | | | mg | g Matrix | | | | (mg) | Matrix |
| | | | SR13668 | material | # | # “00” | | SR13668 | material |
| Formu- | | mg | per | per | persons | capsules | wt. in | needed | needed |
| lation | Matrix used in | SR13668/g | person | person | in study | per | each | for 50 g | for 50 g |
| # | Formulation | matrix | per dose | per dose | (3/group) | person | capsule | batch | batch** |
| 1* | Labrisol/PEG 400 | 5 | 38 | 7.60 | 3 | Unknown* | Unknown* | 250 | 25:25 |
| | (1:1) suspension-Fed | | | | | | | | |
| 1* | Labrisol/PEG 400 | 5 | 38 | 7.60 | 3 | Unknown* | Unknown* | 250 | 25:25 |
| | (1:1) suspension- | | | | | | | | |
| | Fasted | | | | | | | | |
| 2 | Solutol | 5 | 38 | 7.60 | 3 | 8 | 0.95 | 250 | 50 |
| 3 | Solutol:Vit E | 5 | 38 | 7.60 | 3 | 8 | 0.95 | 250 | 25:25 |
| | TPGS (1:1) | | | | | | | | |
| 4 | Vit E TPGS | 5 | 38 | 7.60 | 3 | 8 | 0.95 | 250 | 50 |
| 5 | Myrj 53 | 5 | 38 | 7.60 | 3 | 8 | 0.95 | 250 | 50 |
| | TOTAL | | | | 18 | 32 | | 1500 | |

*Formulation #1 is a suspension at room temperature. At this point the details for dosing this suspension have not been worked out.
**Total Solutol and Vit E TPGS: 75 g each. Total Labrisol and PEG400: 50 g each.

TABLE 7
Stability of SR13668 in Solutol stored as Solid Samples
in Eppendorf tubes under two storage conditions

| | Solid Sample | | | | | | | |
|---|---|---|---|---|---|---|---|---|
| | 25° C. Storage Condition | | | | 4° C. Storage Condition | | | |
| | mg | | | | mg | | | |
| | SR13668/g | | | | SR13668/g | | | |
| Date | Solutol | std | % rsd | n | Solutol | std | % rsd | n |
| 0* | 5.00 | 0.0850 | 1.70 | 5 | 5.00 | 0.0850 | 1.70 | 5 |
| Day | 4.93 | 0.1991 | 4.04 | 3 | 5.05 | 0.0415 | 0.82 | 3 |
| Day 7 | 5.10 | 0.2409 | 4.72 | 3 | 4.97 | 0.0208 | 0.42 | 2 |
| Day 14 | 5.19 | 0.1366 | 2.63 | 3 | 5.33 | 0.1199 | 2.25 | 2 |
| Day 21 | 5.14 | 0.0705 | 1.37 | 3 | 5.06 | 0.1869 | 3.69 | 3 |
| Day 28 | 5.00 | 0.0680 | 1.36 | 3 | 4.97 | 0.0569 | 1.15 | 3 |
| Day 35 | 4.93 | 0.0245 | 0.50 | 3 | 4.87 | 0.0716 | 1.47 | 3 |

*t = 0 for Melt samples, transferred to storage conditions

### Preparation of Samples of SR13668 in Solutol for Dog or Monkey Studies

Sixty, 50-mL centrifuge tubes were filled with 12.1-12.3 g of 5 mg SR13668/g Solutol mixture to supply a dose of 61 mg SR13668 per dog. The samples were made in two batches. Ground SR13668 was added to Solutol melted at 95° C. to give 5 mg/g. Batches were stirred with an overhead stirrer for 48 hrs at 95° C. A small amount water was added to assist the dissolution and then the water was removed under vacuum. Batches assayed at 4.83±0.09 mg/g (n=3) and 5.08±0.11 mg/g (n=3) by HPLC with fluorescence detection.

Oral dosing solutions were prepared by melting the formulation in the centrifuge tube at 65° C. and adding 65° C. water to the 50 mL mark on the tube. The centrifuge tubes were inverted to mix and the formulation easily dissolved.

A similar set of sample tubes was prepared for a monkey study. Twenty-eight tubes with 11.2±0.1 g of 5 mg/g SR13668 in Solutol were prepared providing a dose of 56 mg SR13668 per tube. A set of 3-mL disposable plastic syringes were prepared containing a 5 mg dose of SR13668 in either Solutol, Vit E TPGS or 1:1 Solutol:Vit E TPGS. The syringes were prepared so the dosage form could be pushed out and dissolved in warm water to administer to monkeys by gavage. The syringes were prepared by (a) cutting off the tip-end at the 0.1 mL mark of a plastic, disposable 3-mL syringe, (b) pulling back the plunger, (c) filling the syringe by weight with the liquid melt at 65° C. to give the correct dosage and (d) allowing the formulation to harden. Each formulation contained between 3.5-4.2 mg SR13668/g matrix and approximately 1.2 g of the formulation was needed in each syringe to give a 5 mg dose. The syringes were fairly easy to make and to expel the dosage form into warm water. The doses where shaken in warm water (approximately 40° C.) and took the following amounts of time to dissolve: <1 min. for Solutol, 15 min for 1:1 Solutol:Vit E TPGS, and 37 min for Vit E TPGS.

### Stability of SR13668 in Solutol (1-Month, 25° C. and 4° C.)

A batch of 5 mg SR13668/g Solutol was made by stirring at 95° C. for 24 hrs. No crystals were observed at the end of 24 hrs. The sample was centrifuged while warm and no crystals were observed on the bottom of the tube. The sample was assayed from the top, middle and bottom of the centrifuge tube and there was no significant difference in the measurements. The SR13668 assay is an isocratic HPLC method with Fluorescence detection. The SR13668 concentration in Solutol was determined to be 5.00±0.09 mg/g (n=5).

Three types of samples from the above formulation (a. Solid Samples, b. Aqueous Solution and c. Solid stored and combined with water before assay) were stored at 25° C. (constant temperature bath) and 4° C. (refrigerator) and assayed weekly with the same HPLC/Fluorescence analytical method for 1-month.

Solid Samples

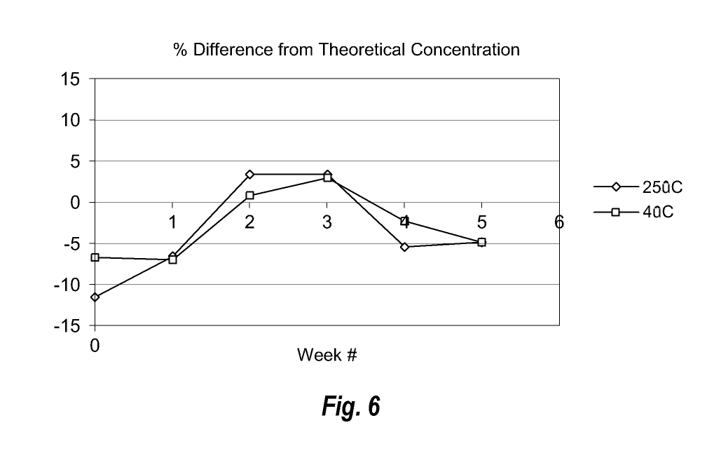

Eppendorf tubes containing 1-mL samples of the above 5 mg SR13668/g Solutol solution were capped and stored under the two temperature conditions described above. At each time point the samples were melted and centrifuged at 95° C. No solids were ever observed in these samples. The analytical results from the stability study are shown in Table 7 and FIG. 4. Samples were stable within the error of the assay and no degradation was observed.

Aqueous Solution

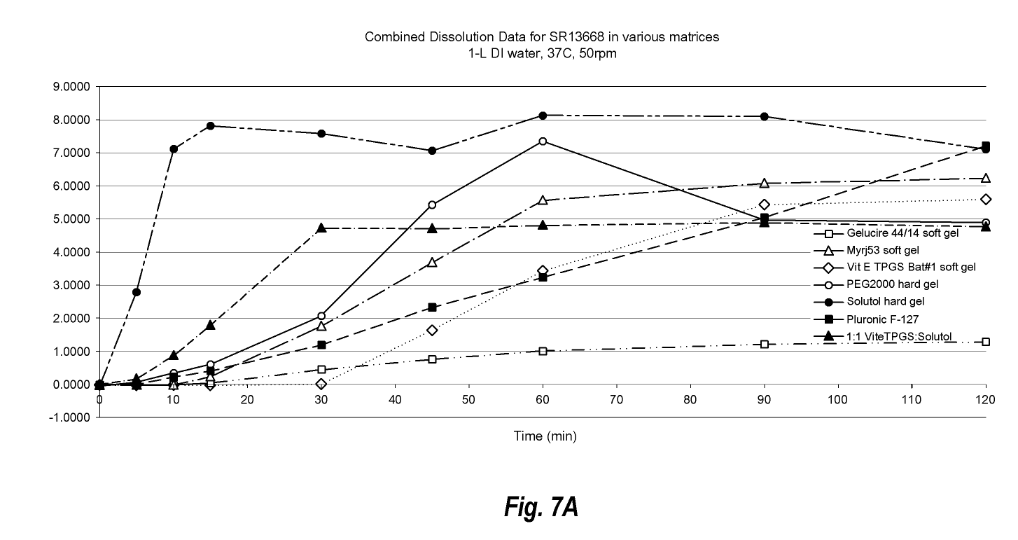

In a 50-mL centrifuge tube, 12 g of the 5 mg SR13668/g Solutol melt was accurately weighed. The tube was tared and water, warmed to 70° C., was added to the 50 mL mark on the tube and weighed. The concentration of SR13668 in the aqueous solution was calculated based on the average SR13668/Solutol concentration of 5.00±0.09 mg/g, the weight of the Solutol and the total weight of solution in the tube. The tube was stored under the two temperature conditions described previously and the assayed amounts were compared with the theoretical. Observations were made on the tubes and there were no significant changes in the solutions and no solids were observed. The 25° C. tube did get a cloudy film layer, possibly biological growth. The layer dispersed after vigorous shaking. The 4° C. samples are cloudy, but clear up when warmed to room temperature. See results Table 8 and FIG. 5. Samples were stable within error and no drug degradation was observed.

TABLE 8
Stability of SR13668 in Solutol stored as aqueous
solution under two storage conditions

| | Aqueous Sample | | | | | | | |
|---|---|---|---|---|---|---|---|---|
| | 25° C. Storage Condition | | | | 4° C. Storage Condition | | | |
| | mg | | | | mg | | | |
| | SR13668/g | | | | SR13668/g | | | |
| Date | Solutol | std | % rsd | n | Solutol | std | % rsd | n |
| 0 | 1.2000 | | | | 1.2000 | | | |
| Day | 1.5040 | 0.1769 | 11.76 | 2 | 1.1384 | 0.0679 | 5.97 | 3 |
| Day 7 | 1.1311 | 0.0237 | 2.09 | 2 | 1.1758 | 0.0198 | 1.68 | 3 |
| Day 14 | 1.2256 | 0.0164 | 1.34 | 2 | 1.1981 | 0.0403 | 3.37 | 3 |
| Day 21 | 1.2663 | 0.0281 | 2.22 | 3 | 1.2509 | 0.0134 | 1.07 | 3 |
| Day 28 | 1.2116 | 0.0055 | 0.46 | 2 | 1.1985 | 0.0065 | 0.54 | 3 |
| Day 35 | 1.1942 | 0.0105 | 0.88 | 3 | 1.1814 | 0.0068 | 0.58 | 2 |

*Theoretical Concentration

Solid Stored and Prepared as Aqueous Solution

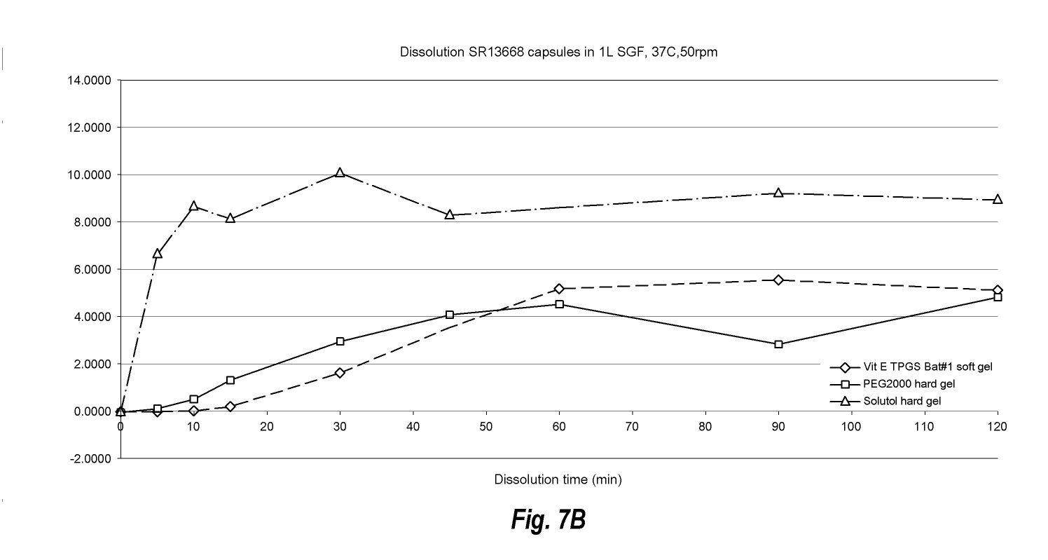

An accurately weighed 200 mg aliquot of the above 5 mg/g Solutol solution was stored at 25° C. and 4° C. At each time point, the solids were melted at 90° C. and 800 mg of accurately weighed 90° C. water was added. The samples were mixed and observations made. In all cases no solids were observed. A sample aliquot of the above aqueous solution was analyzed by HPLC/Fluorescence and the results compared with theoretical. See results Table 9 and FIG. 6. The analytical method is validated ±5% from theoretical. The majority of the samples fell within this limit. As samples at the later time points agree with theory within 5%, there appears to be no trends or indications that the samples are unstable.

TABLE 9
Stability of SR13668 in Solutol stored as solid under
two storage conditions and prepared as aqueous solution

| | Solid/Aq Sample | | | | | |
|---|---|---|---|---|---|---|
| | 25° C. Storage Condition | | | | | |
| | Calculated mg | Measured mg | | | | |
| | SR13668/g | SR13668/g | | | | % difference |
| Date | Solutol | Solutol | std | % rsd | n | from Theory |
| 0* | 0.9751 | 0.8630 | 0.0299 | 3.47 | 3 | −11.50 |
| Day | 1.0654 | 0.9955 | 0.0201 | 2.02 | 3 | −6.56 |
| Day 7 | 0.9971 | 1.0308 | 0.0124 | 1.20 | 3 | 3.38 |
| Day 14 | 0.9294 | 0.9609 | 0.0102 | 1.06 | 3 | 3.40 |
| Day 21 | 0.9914 | 0.9374 | 0.0315 | 3.36 | 3 | −5.45 |
| Day 28 | 0.9849 | 0.9372 | 0.0426 | 4.54 | 3 | −4.84 |

| | Solid/Aq Sample | | | | | |
|---|---|---|---|---|---|---|
| | 4° C. Storage Condition | | | | | |
| | Calculated mg | Measured mg | | | | |
| | SR13668/g | SR13668/g | | | | % difference |
| Date | Solutol | Solutol | std | % rsd | n | from Theory |
| 0* | 0.9902 | 0.9238 | 0.0191 | 2.07 | 3 | −6.71 |
| Day | 0.9468 | 0.8804 | 0.0348 | 3.95 | 2 | −7.00 |
| Day 7 | 0.9790 | 0.9866 | 0.0059 | 0.60 | 3 | 0.78 |
| Day 14 | 1.0308 | 1.0608 | 0.0076 | 0.72 | 2 | 2.91 |
| Day 21 | 1.0039 | 0.9809 | 0.0213 | 2.17 | 3 | −2.29 |
| Day 28 | 0.9630 | 0.9160 | 0.0298 | 3.25 | 3 | −4.87 |

Vit E TPGS, 1:1 Vit E TPGS:Solutol, and Myrj 53

The other three systems are similar to the Solutol solutions, but the Solutol is the best candidate having the lowest freezing point (see Table 10) and a high SR13668 solubility (see Table 11). Solubility data indicates that the 5 mg/g level is feasible in all four formulations (Solutol, Vit E TPGS, 1:1 Vit E TPGS:Solutol, and Myrj 53). The solubility data on these compounds (Table 11) is obtained by over saturating the solution, stirring for several days at 65° C., centrifuging while warm, and analyzing the supernatant. Vit E TPGS is water soluble natural-source vitamin E d-alpha tocopheryl polyethyleneglycol succinate, 387 IU/g. Myrj is Polyethylene Glycol (50) Monostearate.

TABLE 10
Freezing Points of matrices used to solubilize SR13668

| Substance | Freezing Pt. (° C.) (neat) |
|---|---|
| Solutol | 25-30 |
| Vit E TPGS | 37-41 |
| Myrj 53 | 37.2 |

TABLE 11
Solubility of SR13668 in Various Solvents

| | Temperature | | |
|---|---|---|---|
| Solvent | (° C.) | Method | Solubility (ug/mL) |
| Methanol | RT | UV | 86.3 |
| Ethanol | RT | UV | 148 |
| Acetonitrile | RT | HPLC | 84.9 |
| 1-octanol | RT | HPLC | 123.1 |
| DMSO | RT | UV | 106000 |
| Labrasol | RT | UV | 380 |
| Gelucire 44/14 | 65-70 | UV | 4260 |
| PEG-2000 | 65-70 | UV | 8800 |
| PEG-400 | RT | UV | 880 |
| PEG-400 | 65-70 | UV | 9000 |
| Myrj 52 | 65-70 | UV | 6700 |
| Myrj 53 | 65-70 | UV | 6800 |
| Vitamin E TPGS | 65-70 | UV | 5500 |
| PEG-400 New | RT | UV | 1370 |
| PEG-400 New | 65-70 | UV | 7600 |
| PEG-1000 | 65-70 | UV | 8150 |
| Gelucire 50/13 | 65-70 | UV | 4150 |
| Corn Oil | RT | HPLC | 108 |
| Soybean Oil | RT | HPLC | 55 |
| Sesame Oil | RT | HPLC | 47 |
| Sunflower Oil | RT | HPLC | 66 |
| Safflower Oil | RT | HPLC | 77 |
| Water | RT | HPLC | 0.010 |
| Simulated Gastric Fluid | RT | HPLC | 0.014 |
| Simulated Intestinal Fluid | RT | HPLC | 0.0075 |
| Acetone | RT | HPLC | 950 |
| Ethyl Acetate | RT | HPLC | 230 |
| Absolute Ethanol | RT | HPLC | 75 |
| Glycerin | RT | HPLC | 47 |
| Methylene Chloride | RT | HPLC | 62 |
| Propylene Glycol | RT | HPLC | 84 |
| Chemophore EL | RT | HPLC | 264 |
| Solutol | 65-70 | HPLC | 7000 |
| Pluronic F-127 | 120 | HPLC | 9480 |

According to Table 10, solvents having higher than 7000 (e.g., value for Solutol) may be useful for preparing the compositions with the compounds described herein.

### Dissolution Studies

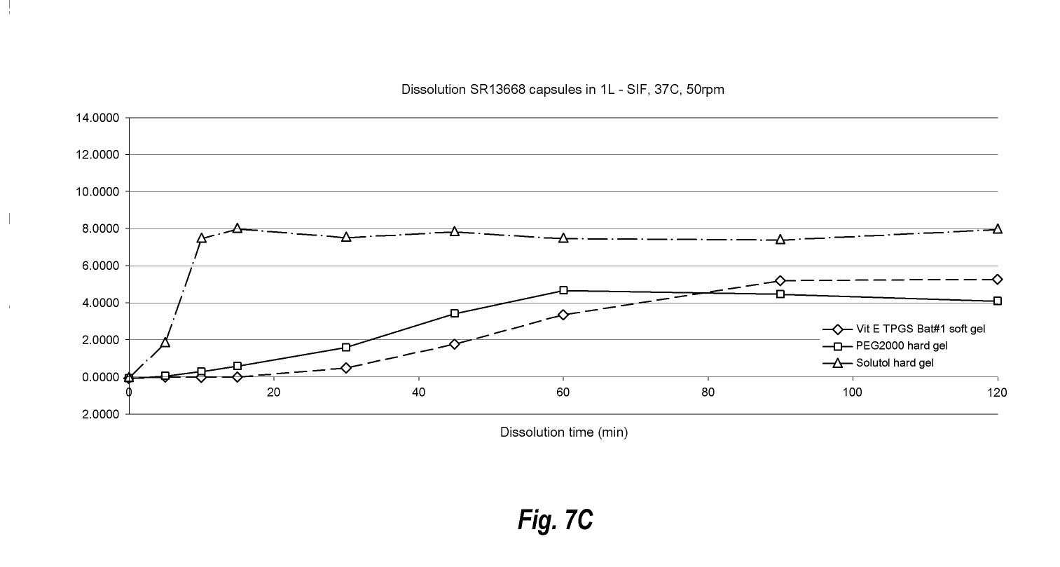

Dissolution studies have been performed on several formulations of SR13668 (see Table 12, FIG. 7A-7C). Different capsules were used, so the initial release profiles are not expected to match. However, from Table 12 it can be seen that in most cases (Formulations A-E) the SR13668 approaches the theoretical concentration at 120 min. It is also noted that the self-emulsifying formulations A-E can be filtered through the 0.45 micron PES filters without complete loss of drug. This is evidence that the drug is encapsulated in micelles keeping it in solution. Some drug is lost as the micelles are large and they may absorb or be partially blocked by the filter. If the drug was present as microcrystals, we would expect it to be filtered out. From the dissolution data, formulations A-E are expected to be good candidates for increased bioavailability of the drug.

Although high concentrations of SR13668 can be achieved in PEG2000 and Pluronic F-127, these non-self emulsifying systems behave differently in dissolution. Both matrices exhibit a slower release profile, as they take longer to dissolve. More importantly, filtering completely removes the drug from the dissolution media. This indicates that the drug is less likely to be in a dissolved state and may not be bioavailable.

TABLE 12
Dissolution Study Summary

| | | | | | | | |
|---|---|---|---|---|---|---|---|
| | | | | Theoretical | Conc. Meas. | Conc. Meas. | Conc. Of |
| | | | | Max. in | at 120 min in | At 24 hrs. In | 24 hr sample |
| | | | Capsule | Dissolution | Dissolution | Dissolution | After |
| | Capsule | Capsule | Assay | Bath* | Bath | Bath | Filtration*** |
| Formulation | Content | Type** | (mgSR13668/g) | (ug/mL) | (ug/mL) | (ug/mL) | (ug/mL) |

Dissolution in H2O

| | | | | | | | |
|---|---|---|---|---|---|---|---|
| A | Gelucire 44/14 | soft gel | 1.40 | 1.46 | 1.28 | 1.28 | 0.96 |
| B | Myrj53 | soft gel | 5.62 | 6.21 | 6.23 | 6.46 | 1.41 |
| C | Vit E TPGS | soft gel | 5.48 | 5.98 | 5.59 | 5.21 | 3.68 |
| D | Solutol | hard gel (000) | 7.83 | 10.93 | 7.11 | 8.00 | 4.71 |
| E | 1:1 Vit E | hard gel (000) | 4.30 | 4.55 | 4.76 | 4.63 | 3.71 |
| | TPGS:Solutol | | | | | | |
| F | Pluronic F-127 | hard gel (000) | 9.48 | 11.20 | 7.21 | 9.71 | 0.00 |
| G | PEG2000 | hard gel (000) | 6.38 | 8.93 | 4.90 | 8.54 | 0.00 |

Dissolution in Simulated Gastric Fluid

| | | | | | | | |
|---|---|---|---|---|---|---|---|
| C | Vit E TPGS | hard gel (000) | 5.48 | 5.98 | 5.12 | 1.13 | 0.25 |
| D | Solutol | hard gel (000) | 7.83 | 10.93 | 8.95 | 8.31 | 3.17 |
| G | PEG2000 | hard gel (000) | 9.48 | 8.93 | 4.83 | 9.47 | 0.00 |

Dissolution in Simulated Intestinal Fluid

| | | | | | | | |
|---|---|---|---|---|---|---|---|
| C | Vit E TPGS | hard gel (000) | 5.48 | 5.98 | 5.29 | 5.03 | 3.23 |
| D | Solutol | hard gel (000) | 7.83 | 10.93 | 7.96 | 6.79 | 3.38 |
| G | PEG2000 | hard gel (000) | 9.48 | 8.93 | 4.10 | 8.69 | 0.00 |

*based on capsule assay and capsule weight
**soft get caps hold approx. 1 g; hard gel “000” caps hold approx. 1.4 g
***sample is filtered through a 0.45 micron PES syringe filter

Additional information regarding indole-3-carbinol compounds and analogs can be found in: U.S. Pat. Nos. 7,429,610; 7,731,776; 7,078,427; 6,800,655; 20090023796; 20080300291; 20060128785; 20040157906; and 20040043965. These indole-3-carbinol compounds can be formulated as described herein.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein by specific reference in their entirety.

1. A composition comprising:
an indole-3-carbinol derivative compound having antitumor activity; and
a pharmaceutically acceptable carrier having the indole-3-carbinol derivative and configured for oral administration so as to provide blood bioavailability of about 0.5% to about 25%.

2. The composition of claim 1, wherein the pharmaceutically acceptable carrier includes a hydroxyl-fatty acid PEG monoester and/or diester.

3. The composition of claim 1, wherein the carrier is a hydroxyl-fatty acid PEG ester that includes 12-hydroxy stearate.

4. The composition of claim 1, wherein the carrier is a hydroxyl-fatty acid PEG ester that includes a PEG having from about 100 MW to about 200,000 MW.

5. The composition of claim 1, wherein the indole-3-carbinol derivative is 2,10-dicarbethoxy-6-methoxy-5,7-dihydro-indolo-(2,3-b)carbazole.

6. The composition of claim 1, wherein the indole-3-carbinol derivative is present from about 0.5 mg to about 15 mg per gram of pharmaceutically acceptable carrier.

7. The composition of claim 1, wherein the indole-3-carbinol derivative is 2,10-dicarbethoxy-6-methoxy-5,7-dihydro-indolo-(2,3-b)carbazole and is present up to about 13 mg per gram of pharmaceutically acceptable carrier.

8. The composition of claim 1, wherein the pharmaceutically acceptable carrier includes free PEG.

9. The composition of claim 1, wherein the pharmaceutically acceptable carrier includes free PEG up to about 50%.

10. The composition of claim 1, wherein the composition is a dose that contains from about 10 mg to about 100 mg of the indole-3-carbinol derivative.

11. The composition of claim 1, wherein the composition is a dose in the form of a gel capsule.

12. A method of manufacturing a composition of claim 1, the method comprising:
obtaining powdered and/or crystalline indole-3-carbinol derivative; and
combining the crystalline indole-3-carbinol derivative with the pharmaceutically acceptable carrier under heat and stifling to form a mixture.

13. The method of claim 12, comprising grinding crystalline indole-3-carbinol derivative into a powder.

14. The method of claim 13, comprising heating the mixture to at least about 65° C.

15. The method of claim 14, comprising heating the mixture to less than about 110° C.

16. The method of claim 13, comprising heating the mixture to between about 65° C. to about 95° C.

17. The method of claim 13, comprising configuring the mixture into an oral formulation having the bioavailability.

18. The method of claim 13, comprising filling a capsule with the mixture.

19. A method of treating, inhibiting, and/or preventing cancer, the method comprising:
orally administering the composition of claim 1 to a subject.

20. (canceled)

21. (canceled)

22. (canceled)

23. The method of claim 19, comprising administering a therapeutically effective amount of the composition in order to treat, inhibit, and/or prevent cancer.
